(19) United States
(12) Patent Application Publication
(10) Pub. No.: US 2026/0294240 A1
(43) Pub. Date: Oct. 1, 2026

(54) NON-CONTACT WIDE ANGLE VIEWING SYSTEM WITH FRONT LENS ASSEMBLY HAVING ANTI-FOGGING, ANTI-REFLECTIVE COATING

(71) Applicant: Alcon Inc., Fribourg (CH)

(72) Inventors: Chenguang Diao, Irvine, CA (US); Christopher Paul Voita, Dove Canyon, CA (US); Sumit Paliwal, Irvine, CA (US)

(21) Appl. No.: 19/567,877

(22) Filed: Mar. 16, 2026

Related U.S. Application Data
(60) Provisional application No. 63/778,816, filed on Mar. 27, 2025.

(51) Int. Cl. A61B 3/13; G02B 1/04; G02B 1/11; G02B 1/18; G02B 7/02; G02B 21/00

(52) U.S. Cl. CPC A61B 3/132; G02B 1/041; G02B 1/11; G02B 1/18; G02B 7/023; G02B 21/0012

(57) ABSTRACT

A front lens assembly for a non-contact wide angle viewing system (WAVS) connected to an ophthalmic microscope includes a front lens having a first surface configured to face an eye of patient during an ophthalmic surgery, and a second surface configured to face the microscope when the front lens assembly is connected thereto. An anti-fogging, anti-reflective (AFAR) layer coats at least the first surface and prevents an accumulation of fog on the front lens during the surgery. The AFAR layer also may coat the second surface. The WAVS may include an adjustable bracket having a first end that is pivotably connectable to the microscope and a second end that is fixedly connectable to the lens. An anti-fogging layer may coat some or all of the bracket to prevent an accumulation of the fog on the bracket during the surgery.

### INTRODUCTION

The present disclosure relates to an indirect/non-contact wide angle viewing systems (WAVS) having a front lens assembly for viewing a patient's ocular anatomy under various levels of magnification.

Vitreoretinal surgery involves the performance of delicate surgical tasks in and around the eye's fundus region. The prognosis and diagnosis of injuries, diseases, and other ocular conditions often requires a surgeon to employ an ophthalmic microscope for high-definition visualization. A WAVS is connected to or integrated with the microscope to provide a magnified real-time view of the fundus region, with optional image capture provided by a digital camera. In this manner, the surgeon is afforded a clear view of the retina, macula, vitreous humor, and surrounding tissue within the eye.

During an ophthalmic visualization procedure, a surgeon may require a wider view of the fundus region than is ordinarily achievable using the microscope's internal lenses. For instance, the surgeon might find it beneficial to view the peripheral retina area when monitoring for retinal tears or detachments. To this end, a WAVS uses a specially-constructed ocular lens, which in some implementations is placed directly on the patient's cornea as a contact lens. In contrast to direct WAVS, indirect/non-contact WAVS implementations position a magnifying front lens a short distance away from the patient's cornea. The construction of the front lens in either instance provides a beneficial wide angle view of the fundus region.

### SUMMARY

Disclosed herein are an indirect/non-contact wide angle visualization system (WAVS) having a front lens assembly, parts of which are coated with an anti-reflective, anti-fogging material for minimizing condensation and resulting fog on the treated surfaces. As appreciated in the art, a front lens of a non-contact WAVS provides the widest possible view of the above-summarized fundus region when the front lens is positioned in close proximity to the cornea surface, typically within a few millimeters. However, the patient's normal respiration and other factors such as the temperature and relative humidity level of the operating room may cause moisture in the air to condense on the front lens assembly, including the lens's inner surface closest to the patient's eye. The accumulation of fog may require the surgeon or attending operating room staff to periodically lift and clean the front lens. To prevent fogging of the front lens, the surgeon might also decide to position the front lens a bit farther from the cornea surface. However, doing so reduces the viewing angle and may impair the retina imaging quality. The solutions set forth herein are therefore intended to improve upon the general state of the art of non-contact WAVS by preventing formation of condensation/fog on the front lens, with the present teachings possibly being extendable to other types of ophthalmic lenses.

In accordance with one or more non-limiting configurations, a front lens assembly is disclosed herein for a non-contact WAVS that is connectable to an ophthalmic microscope. The WAVS includes a magnifying front lens and an anti-fogging, anti-reflective (AFAR) layer. The front lens may be constructed of molded plastic or glass in different implementations, e.g., as a disposable or autoclavable lens, respectively.

A first surface of the lens faces an eye of patient during an ophthalmic surgery performed using the WAVS. A second surface of the lens is configured to face the microscope when the WAVS is connected to the microscope, e.g., via an adjustable arm or bracket. The AFAR layer, which is disposed on at least the first surface, is configured to prevent accumulation of fog on the magnifying lens and possibly other surfaces of the front lens assembly during the surgery.

The front lens assembly may include the adjustable bracket, which in turn has opposing first and second ends. The first end of the bracket is pivotably connectable to the microscope. The second end is fixedly connectable to an outer perimeter of the front lens. The adjustable bracket in one or more implementations may include an annular connection ring disposed at the second end. In such an embodiment, the annular connection ring may be coated with an anti-fogging (AF) layer, with the AF layer being configured to prevent an accumulation of the fog on the connection ring.

The AFAR layer(s) may include a hydrophobic material, a hydrophilic material, or mixture of both materials in different constructions.

Also disclosed herein is a non-contact WAVS having a front lens assembly. The front lens assembly in turn may include an adjustable bracket and a magnifying front lens. The front lens may include an outer perimeter connected to the adjustable bracket, a first surface that faces an eye of a patient during an ophthalmic surgery performed using the WAVS, and a second surface that faces the ophthalmic microscope. An AFAR layer, which is disposed on/coats at least the first surface, is configured to prevent an accumulation of fog on the front lens during the ophthalmic surgery.

Embodiments of the non-contact WAVS may include a processor in communication with the microscope, and a computer-readable storage medium (“memory”) on which is recorded instructions. Execution of the instructions by the processor causes the processor to determine an identity of the front lens assembly, the identity including a characteristic of the AFAR layer, and adjust a fluence level of the microscope based on the identity of the front lens assembly.

The non-contact WAVS may also include a scanning tool in communication with the processor. The scanning tool, e.g., a barcode or QR code scanner, is operable for scanning a code that is indicative of the identity of the front lens assembly. The processor in such an embodiment is configured to determine the identity of the front lens assembly by processing an electronic signal from the scanning tool that is indicative of the identity, e.g., a unique serial number, model, or other indicator.

Another embodiment of the lens assembly for the non-contact WAVS summarized above includes a plastic magnifying lens having a circular outer perimeter, a first convex surface configured to face an eye of patient during an ophthalmic surgery performed using the WAVS, and a second convex surface configured to face the reduction lens assembly attached to/mounted on the microscope when the front lens assembly is connected thereto. The non-contact WAVS also includes an adjustable bracket having a first end that is pivotably connectable to the reduction lens assembly attached to/mounted on the microscope, and a second end forming a connection ring that is coupled to the outer perimeter. A first AFAR layer is disposed on/coats the first and second convex surfaces in this non-limiting embodiment, and is configured to prevent an accumulation of fog on the front lens during the surgery. A second AFAR layer is disposed on/coats the connecting ring and is configured to prevent an accumulation of the fog thereon during the ophthalmic surgery. The first AFAR layer and the second AFAR layer may include a mixture of hydrophobic and/or hydrophilic materials.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 illustrates an exemplary ophthalmic surgical suite equipped with a system having an ophthalmic microscope connected to a non-contact wide-angle visualization system (WAVS), with a front lens assembly of the WAVS having one or more surfaces coated with an anti-fogging, anti-reflective (AFAR) layer in accordance with the present disclosure.

FIG. 2 is an illustration of the front lens assembly shown in FIG. 1 in use during an ophthalmic surgery.

FIGS. 3A, 3B, and 3C are partial cross-sectional side views of different portions of the front lens assembly illustrated in FIG. 2.

FIG. 4 is a perspective view illustration of the front lens assembly of FIG. 2.

FIG. 5 is a flow chart describing a method for controlling the microscope of FIG. 1 based on an identity of the front lens assembly.

The solutions of the present disclosure may be modified or presented in alternative forms. Representative embodiments are shown by way of example in the drawings and described in detail below. However, inventive aspects of this disclosure are not limited to the disclosed embodiments. Rather, the present disclosure is intended to cover alternatives falling within the scope of the disclosure as defined by the appended claims.

### DETAILED DESCRIPTION

Embodiments of the present disclosure are described in detail herein. Disclosed embodiments are provided as examples, with other embodiments possibly taking alternative forms. The Figures are not necessarily drawn to scale. For instance, some features could be exaggerated or minimized to show details of particular components. Therefore, specific structural and functional details disclosed herein are not to be interpreted as limiting, but merely as a representative basis for teaching one skilled in the art to employ the present disclosure.

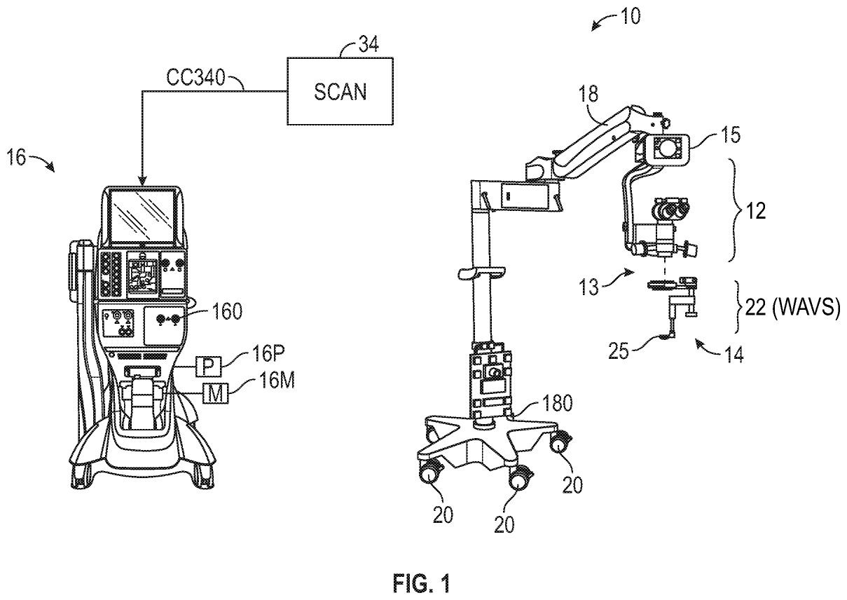

Referring to the drawings, wherein like reference numbers refer to like components, a system 10 having an ophthalmic microscope 12, a surgical console 16, and a support arm 18. A non-contact wide angle visualization system (WAVS) 22 is connected to the microscope 12 as illustrated in FIG. 1. The WAVS 22 may include a wide angle front lens assembly 14 constructed as set forth below. The WAVS 22 in one or more embodiments may also include or communicate with the surgical console 16 and the support arm 18, e.g., a robotic or non-robotic, multi-axis device, with the support arm 18 operatively connected to a base 180 in a possible construction. For mobility within an operating room, the base 180 may be equipped with a set of lockable wheels 20.

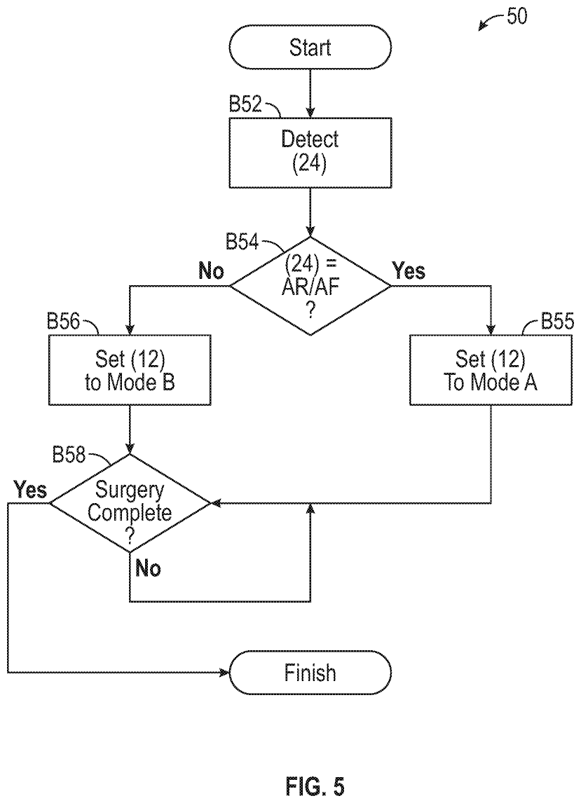

As set forth in detail hereinbelow, select surfaces of the front lens assembly 14 are coated with an anti-reflective, anti-fogging (AFAR) material to prevent accumulation of moisture/fog on the front lens assembly 14. Other aspects of the disclosure pertain to controlling operation of the microscope 12 based on the presence of such AFAR materials on the front lens assembly 14, with a representative method 50 for doing so described below with reference to FIG. 5.

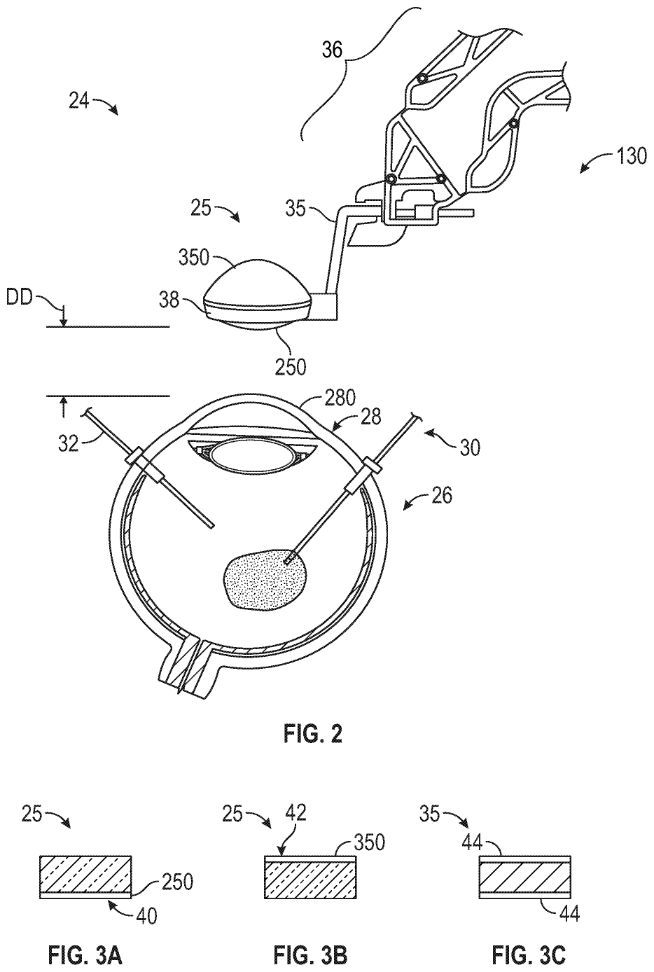

In the illustrated configuration, the microscope 12 is connected to the support arm 18. The front lens assembly 14 of the WAVS 22 in turn is removably connectable to the microscope 12 via an adjustable connecting portion 13, e.g., the commercially available ZEISS Resight™ Fundus Imaging System or OCULUS BIOM®. The front lens assembly 14 is modified herein to include an anti-fogging, anti-reflective (AFAR) front lens 25 as described in further detail below with reference to FIGS. 2-5. As noted above, an ocular lens of an indirect/non-contact WAVS, exemplified herein by the front lens 25 of the non-contact WAVS 22, respectively, provides a surgeon with wide angle views of the fundus region of a patient's eye 26 (see FIG. 2) when used in conjunction with the microscope 12. Viewing of the eye 26 during surgery may also be achieved using one or more high-resolution display screens 15 and oculars 150, as appreciated in the art.

Ophthalmic surgical consoles such as the surgical console 16 of FIG. 1 are typically equipped with multiple columns and rows of connection ports 160 providing the requisite electrical, data, pressure, irrigation, suction, and other power connections needed for supporting a given surgical procedure and its related surgical tools. The surgical console 16 may be configured as or include an electronic control unit in communication with other systems or components, e.g., via a wired or wireless communications network or individual transfer conductors. The surgical console 16 may also include one or more processors (P) 16P and sufficient computer-readable storage media/tangible-not transitory memory (M) 16M, e.g., optical, magnetic, flash, or other types of read only memory, along with application-sufficient amounts of random-access memory, electrically-erasable programmable read only memory, etc. The processor(s) for their part may be constructed from various combinations of Application Specific Integrated Circuit(s) (ASICs), Field-Programmable Gate Arrays (FPGAs), electronic circuits, central processing units, microprocessors, and the like.

In some configurations, the surgical console 16 and its resident processor 16P may be operable for receiving an electronic signal (CC₃₄₀) from a scanning device 34, e.g., a hardwired or wirelessly-connected bar code scanner, QR code scanner/camera, or other device operable for scanning a digitized pattern, binary, or alphanumeric code that is indicative of the identity of the front lens assembly 14. Execution by the processor 16P of algorithm code/computer-readable instructions stored in the memory 16M cause the processor 16P in one or more representative embodiments to determine an identity of the front lens assembly 14, for instance its manufacturer, make and model, year of manufacture, etc. The information may be used as part of a method 50 for controlling the WAVS 22, with a non-limiting implementation of the method 50 described below with reference to FIG. 5.

Referring now to FIG. 2, the eye 26 is depicted undergoing a vitrectomy surgery with the wide angle viewing assistance of the WAVS 22 (FIG. 1). Representative surgical tools 30 and 32 are shown penetrating the eye 26 and performing an operation within the vitreous cavity 29. During vitrectomy surgery, the front lens 25 of the front lens assembly 14 is positioned via adjustment of the microscope 12 and/or the connecting portion 13 of FIG. 1 until the front lens 25 is predetermined a standoff distance (DD) from a cornea surface 28, with a standoff distance (DD) of about 5-10 millimeters (mm) being typical. The front lens 25 is therefore “non-contact” in the sense that the front lens 25 does not physically contact the cornea surface 28 during surgery, contrary to a contact-type WAVS. Rather, vertical motion of the microscope 12 or adjustable connecting portion 13, along with replacement of the front lens 25 with higher or lower diopter variants, effectively controls magnification and field of view levels.

Absent the present teachings, the patient's breath, low room temperature, relatively cool surfaces of the front lens assembly 14, and ambient humidity may at times cause water vapor to condense as an opaque film or fog on a first surface 250 of the front lens 25, i.e., a lower (convex) surface of the front lens 25 located closest to the cornea surface 28, and thus facing the eye 26. Fogging may require the surgeon or attending medical staff (not shown) to increase the standoff distance (DD) in an attempt at reducing fogging of the front lens 25, and/or to periodically lift the front lens 25 to wipe the first surface 250 and clear it of accumulated condensation. Lens fogging may also occur to some extent on an opposing second surface 350 of the front lens 25, i.e., the particular surface (also convex as in the illustrated embodiment) configured to face the microscope 12 of FIG. 1 when the front lens assembly 14 is connected thereto. Fogging may also occur on an adjustable bracket 35 of the front lens assembly 14, for instance a metal bracket terminating in a connection ring 38. The bracket 35 is thus configured to connect the front lens 25 to the microscope 12 (FIG. 2), which occurs via an intervening adjustable connecting portion 130, e.g., a hinged lattice structure as shown, or the adjustable connecting portion 13 of FIG. 1 or another suitable connection.

In general, condensation and resulting fogging of the front lens 25 of FIG. 2 may occur whenever water vapor contacts the front lens assembly 14. This is particularly true in colder operating rooms. In general, surface tension causes condensed water droplets to adhere to the respective first and second surfaces 250 and 350, and possibly to surfaces of the adjustable bracket 35 and ring 38. While condensation on the bracket 35 or ring 38 may not immediately interfere with a surgeon's visibility of the eye 26 through the front lens 25, the condensed water may eventually coalesce and trickle along the bracket 35 via gravity, eventually contacting the front lens 25. To that end, the front lens assembly 14 is treated herein to minimize the accumulation of fog on its first surface 250, and possibly its second surface 350 and/or the bracket 35.

In addition to being anti-fogging, the coatings used herein on the front lens assembly 14 are anti-reflective. It is possible that the application of anti-fogging hydrophobic and/or hydrophilic materials may result in reflections, their presence of which may interfere with visibility of the eye 26 when views through the front lens 25. Anti-reflective structure such as microscopic (nanometer scale) surface asperities may be used to create destructive interference and allow light to pass through the front lens 25. As appreciated by those skilled in the art, the index of refraction of metal oxides of varying refractive indices, or other application suitable AR materials used to coat the front lens 25, may lie somewhere between that of air and the materials of the front lens 25. The construction of the stack layers of AR materials ensures that reflected light waves from the AR layer are 180° out of phase with light waves reflected from the underlying optical surface of the front lens 25. Reflected light waves thus undergoes the above-noted destructive interference and effectively cancels out, providing a clear view of the eye 26 through the front lens.

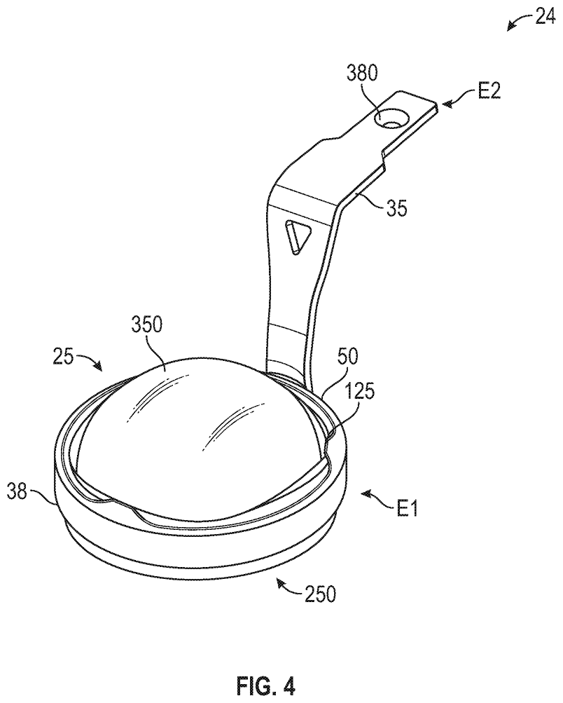

Referring briefly to FIGS. 3A and 3B, a cross-section portion of the front lens 25 is shown to depict coating of the first surface 250 (FIG. 3A) and second surface 350 (FIG. 3B) with an anti-fogging, anti-reflective (AFAR) layer 40 and 42, respectively. FIG. 3C illustrates similar coating of the bracket 35 with an anti-fogging (AF) layer 44. Note that AF layer 44 in this instance may forego use of anti-reflective materials, as the bracket 35 is neither located in the beam path nor transmissive. In different embodiments, the AFAR layers 40 and 42, and the AF coating 44, may be constructed as a thin coating of hydrophobic and/or hydrophilic material. Representative materials suitable for use in the surgical environment of FIG. 1, which may be applied during production using a deposition process, may include a single layer of (or a combination of layers of) magnesium fluoride (MgF2), silicon dioxide (SiO2, tantalum oxide (Ta2O5), zirconium oxide (ZrO2), and/or hafnium oxide (HfO2) in different combinations, with the materials or other materials, e.g., engineered polymers or metal oxides, likewise reducing reflections.

In the ophthalmic surgical use cases contemplated herein, the use hydrophobic AR materials, hydrophilic AR materials, or a mixture of both is intended to improve the state of the art for usability in an ophthalmic surgical context by altering surface tension and interactions between water molecules and the treated surfaces, i.e., AFAR layers 40 or 42 or AF layer 44 of FIGS. 3A, 3B, and 3C, respectively. A hydrophobic coating would tend to repel water such that the water forms droplets. The high contact angle in turn allows the coalesced droplets to run off the front lens 14 or bracket 35. In contrast, use of a hydrophilic coating would tend to cause water to spread evenly across the treated surfaces of the front lens assembly 14 as a thin, transparent layer, with no resulting image degradation in the case of the AFAR layers 40 and 42. This is due to the water no longer being in the form of discrete liquid droplets.

Referring to FIG. 4, the front lens assembly 14 is shown in perspective view in accordance with an embodiment in which the bracket 35 is a generally L-shaped metal bracket having respective first and second ends E1 and E2. Connection ring 38 is integrally formed with or attached to the first end E1. A planar tab portion 39 (possibly with one or more mounting holes 390) is disposed at the second end E2. The connection ring 38 in the illustrated construction is fixedly connected to an outer perimeter 125 of the front lens 25, e.g., via radial tabs 38T. The outer perimeter 125 may be a circular outer perimeter in certain embodiments of the front lens 25, or the outer perimeter 125 may have a multi-faceted, non-circular, or other application suitable shape.

In the illustrated construction, the connection ring 38 forms a complete annulus or ring that fully circumscribes the front lens 25. The connection ring 38 may be constructed from a wide range of application-suitable sterile materials. By way of a few illustrative examples, the connection ring 38 may be optionally constructed of a metal such as stainless steel or aluminum, a metal alloy such as but not limited to nitinol, or a molded plastic or elastomeric material such as an application-suitable polymer, PVC, or a flexible shape-memory alloy (SMA). In another construction, to reduce stack-up tolerance, the entire front lens assembly 14, i.e., the front lens 25, connection ring 38, and adjustable bracket 35 having ends E1 and E2, can be integrally formed or molded as a single piece using plastic materials, e.g., polymethyl methacrylate (PMMA), i.e., acrylic.

Referring to FIG. 5, a method 50 is described in terms of discrete process steps, segments, or logic blocks for clarity. Some of the hardware solutions set forth above may be implemented in software, for example by programming the memory 16M of the surgical console 16 shown in FIG. 1 with computer-readable instructions or algorithms/code segments, or logic blocks, the execution of which by the processor 16P causes the processor 16P to perform the noted actions. Thus, a corresponding automated routine may initialize (“Start”) with commencement of a vitrectomy or other surgical procedure of the eye 26 show in FIG. 2.

Beginning with block B52 (“Detect (14)”) includes detecting the front lens assembly 14. As described above, the optional scanning tool 34 may be placed in communication with the processor 16P or another control device, with the scanning tool 34 being operable for scanning a code that is indicative of the identity of the front lens assembly 14. The front lens assembly 14 may be delivered to the operating room in a sterile package having a code printed thereon or connected thereto, e.g., as a sticker or label, and/or such a code may be printed on/attached to the front lens assembly 14. Detecting the front lens assembly 14 may therefore entail receiving the electronic signal (CC₃₄₀) of FIG. 1 in response to a scanning operation, e.g., as a bit string or voltage signal indicative of the identity. The method 50 thereafter proceeds to block B54.

Block B54 (“AFAR?”) includes determining an identity of the front lens assembly 14. In a possible embodiment, the identity includes an identifying characteristic of the front lens assembly 14. Block B54 may therefore entail processing the electronic signal (CC₃₄₀) from the scanning tool 34 to determine the characteristic, e.g., a make or model of the front lens assembly 14. The processor 16P may then compare the characteristic of the front lens assembly 14 to a lookup table to determine whether the front lens 25 and/or the bracket 35 have been treated with the AFAR layer 40, 42, or the AF layer 44 of FIGS. 3A, 3B, or 3C.

Block B54 may also include determining characteristics of the AFAR layer(s) 40, 42, and/or the AF layer 44 to ascertain effects of the layers(s) 40, 42, and/or 44 on light transmission, viewing clarity, or other factors. The method 50 then proceeds to block B55 when the processor 16P has determined that the front lens assembly 14 has been treated with the layer(s) 40, 42, and/or 44, along with the characteristics of the layer(s) 40, 42, and/or 44. The method 50 proceeds in the alternative to block B55 when the processor 16P has determined that the front lens assembly 14 has not been treated with the layer(s) 40, 42, and/or 44.

At block B55 (“Set (12) to Mode A”), the processor 16P may command or set a first mode, i.e., nominal Mode A, via communication with processors (not shown) of the microscope 12. In doing so, the processor 16P may adjust a control parameter and output state of the microscope 12. For instance, block B55 may entail adjusting a fluence level or other setting of the microscope 12 based on the identity of the front lens assembly 14/front lens 25. The method 50 proceeds to block B58 after the adjustment has been made.

At block B56 (“Set (12) to Mode B”), the processor 16P may command or set a second mode, i.e., a default Mode B, via communication with processors (not shown) of the ophthalmic microscope 12. In doing so, the processor 16P may maintain a control parameter and output state of the microscope 12, doing so in response to a determination at block B54 that the front lens assembly 14 has not been treated with the AFAR layers 40, 42, or 44 of FIGS. 3A, 3B, or 3C, respectively. The method 50 proceeds to block B58.

Block B58 (“Surgery Complete?”) includes determining if the surgery is complete. Block B58 continues in a loop until such time as the microscope 12 is shut off or the surgeon otherwise signals the end of the surgery. If at any point during surgery the front lens assembly 14 should be replaced, such an action may terminate method 50, which would thereafter commence anew at block B52. The method 50 is finished when the surgery has been completed.

The present solutions thus envision treating one or more surfaces of the front lens assembly 14 of FIGS. 1, 2, and 4 with an AFAR coating to reduce accumulation of fog on the front lens 25 and/or bracket 35. Embodiments described herein provide for disposable constructions of the front lens assembly 14, along with reusable, autoclavable/sterilizable constructions. To the limited extent that treatment of the front lens assembly 14 with the AFAR coating affects viewing properties when using the front lens 25, the method 50 may be used to provide a control loop to the microscope 12 to adjust fluence levels or other parameters as needed. Improved viewing clarity and reduced need for intervention during surgery are some of the benefits of the present non-fogging solutions when applied to indirect/non-contact wide-angle visualization, as will be readily appreciated by those skilled in the art now having the benefit of the foregoing disclosure.

As those of ordinary skill in the art will understand, various features illustrated and described with reference to any one of the Figures can be combined with features illustrated in one or more other Figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Embodiments shown in the drawings or the characteristics of various embodiments mentioned in the present description are not necessarily to be understood as being independent of each other. It is possible that each of the characteristics described in a given embodiment could be combined with one or more other desired characteristics from other embodiments, resulting in other embodiments not described in words or by reference to the drawings. Accordingly, such other embodiments fall within the framework of the scope of the appended claims.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

We claim:

1. A front lens assembly for a non-contact wide angle viewing system (WAVS) connected to an ophthalmic microscope, the front lens assembly comprising:
a front lens having an outer perimeter, a first surface configured to face an eye of a patient during an ophthalmic surgery performed using the WAVS, and a second surface configured to face the ophthalmic microscope when the front lens assembly is connected to the ophthalmic microscope; and
an anti-fogging, anti-reflective (AFAR) layer that coats at least the first surface, the AFAR layer being configured to prevent an accumulation of fog on at least the first surface of the front lens during the ophthalmic surgery.

2. The front lens assembly of claim 1, wherein the AFAR layer is attached to or coats the second surface, and is configured to prevent an accumulation of fog on the second surface of the front lens during the ophthalmic surgery.

3. The front lens assembly of claim 1, further comprising:
an adjustable bracket having:
a first end that is fixedly connectable to the outer perimeter; and
a second end that is pivotably connectable to the ophthalmic microscope.

4. The front lens assembly of claim 3, wherein the adjustable bracket includes a connection ring disposed at the first end, further comprising:
an anti-fogging (AF) layer that coats the connection ring to prevent an accumulation of fog on the connection ring during the ophthalmic surgery.

5. The front lens assembly of claim 1, wherein the front lens is constructed of a molded plastic.

6. The front lens assembly of claim 1, wherein the front lens is constructed of glass.

7. The front lens assembly of claim 1, wherein the AFAR layer includes a hydrophobic material.

8. The front lens assembly of claim 1, wherein the AFAR layer includes a hydrophilic material.

9. A system comprising:
an ophthalmic microscope; and
a non-contact wide angle viewing system (WAVS) connected to the ophthalmic microscope, the WAVS including
a front lens assembly comprising:
an adjustable bracket;
a front lens having an outer perimeter connected to the adjustable bracket, a first surface configured to face an eye of a patient during an ophthalmic surgery performed using the WAVS, and a second surface configured to face the ophthalmic microscope during the ophthalmic surgery; and
an anti-fogging, anti-reflective (AFAR) layer that coats at least the first surface and is configured to prevent an accumulation of fog thereon during the ophthalmic surgery.

10. The system of claim 9, wherein the adjustable bracket includes:
a first end that is connectable to the outer perimeter of the front lens; and
a second end that is pivotably connected to the ophthalmic microscope.

11. The system of claim 9, wherein the AFAR layer additionally coats the second surface.

12. The system of claim 9, wherein the adjustable bracket includes a connection ring disposed at a first end, further comprising:
an anti-fogging (AF) layer that coats the connection ring and is configured to prevent an accumulation of the fog thereon during the ophthalmic surgery.

13. The system of claim 9, wherein the front lens is constructed of a molded plastic.

14. The system of claim 9, wherein the AFAR layer includes a hydrophobic material.

15. The system of claim 9, wherein the AFAR layer includes a hydrophilic material.

16. The system of claim 9, further comprising:
a processor in communication with the ophthalmic microscope;
a computer-readable storage medium (“memory”) on which is recorded instructions, wherein execution of the instructions by the processor causes the processor to:
determine an identity of the front lens assembly, the identity including a characteristic of the front lens assembly indicative of a presence of the AFAR layer; and
adjust a fluence level of the ophthalmic microscope based on the identity of the front lens assembly.

17. The system of claim 16, further comprising:
a scanning tool in communication with the processor, the scanning tool being operable for scanning a code that is indicative of the identity of the front lens assembly, wherein the processor is configured to determine the identity of the front lens assembly by processing an electronic signal from the scanning tool that is indicative of the identity.

18. A front lens assembly for a non-contact wide angle viewing system (WAVS) connectable to an ophthalmic microscope, the front lens assembly comprising:
a disposable plastic front lens having a circular outer perimeter, a first convex surface configured to face an eye of patient during an ophthalmic surgery performed using the WAVS, and a second convex surface configured to face the ophthalmic microscope when the front lens assembly is connected to the ophthalmic microscope;
an adjustable bracket having a first end that is pivotably connectable to the ophthalmic microscope, and a second end forming a connection ring that is fixedly connectable to the circular outer perimeter;
a first anti-fogging, anti-reflective (AFAR) layer and a second AFAR layer, wherein the first AFAR layer coats the first convex surface and the second AFAR layer coats the second convex surface to respectively prevent an accumulation of fog on the first surface and the second surface during the ophthalmic surgery,
wherein the first AFAR layer and the second AFAR layer each include a mixture of a hydrophobic material and a hydrophilic material.

19. The front lens assembly of claim 18, further comprising:
an anti-fogging (AR) layer that coats the connection ring and is configured to prevent an accumulation of the fog on the connection ring during the ophthalmic surgery.

20. The front lens assembly of claim 18, wherein the disposable plastic front lens and the adjustable bracket are integrally formed from acrylic.
